(19) United States
(12) Patent Application Publication
(10) Pub. No.: US 2026/0055104 A1
(43) Pub. Date: Feb. 26, 2026

(54) CRYSTAL FORM OF HETEROARYL DERIVATIVE PARP INHIBITOR AND USE THEREOF

(71) Applicant: HAISCO PHARMACEUTICAL GROUP CO., LTD., ShannanCIty, Tibet (CN)

(72) Inventors: Zheng GONG, Chengdu, Sichuan (CN); Jinyi MA, Chengdu, Sichuan (CN); Xianhua DENG, Chengdu, Sichuan (CN); Jiang FAN, Chengdu, Sichuan (CN); Ying DOU, Chengdu, Sichuan (CN)

(21) Appl. No.: 19/106,110

(22) PCT Filed: Aug. 24, 2023
(86) PCT No.: PCT/CN2023/114695

§ 371 Date: Feb. 24, 2025

(30) Foreign Application Priority Data
Aug. 24, 2022 (CN) 202211016469.5
Mar. 13, 2023 (CN) 202310238294.0

(51) Int. Cl. C07D 471/04; A61K 31/496; A61P 35/00

(52) U.S. Cl. CPC C07D 471/04; A61K 31/496; A61P 35/00

(57) ABSTRACT

The present invention relates to a crystal form of a compound N-cyclopropyl-5-(4-((7-ethyl-6-oxo-5.6-dihydro-1.5-naphthyridin-3-yl)methyl)piperazin-1-yl)pyridine carboxamide and a preparation method therefor, and the use thereof in the preparation of a related drug.

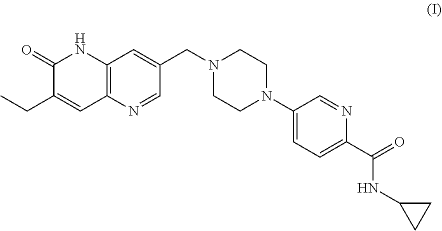 [SMILES: CCc1cc2ncc(CN3CCN(c4ccc(C(=O)NC5CC5)nc4)CC3)cc2[nH]c1=O]

### CROSS-REFERENCE TO RELATED APPLICATION

This application is a 35 U.S.C. § 371 National Stage of International Patent Application No. PCT/CN2023/114695, filed Aug. 24, 2023, designating the United States, which claims priority to and the benefits of Chinese Patent Application No. 202211016469.5, filed Aug. 24, 2022, Chinese Patent Application No. 202310238294.0, filed Mar. 13, 2023, the disclosures of which are incorporated herein in their entirety by reference, and priority is claimed to each of the foregoing.

### TECHNICAL FIELD

The present invention belongs to the field of drugs, and in particular relates to a plurality of crystal forms of a small molecule compound having inhibitory activity against PARP-1, and the use thereof in the preparation of a drug for treating a related disease.

### BACKGROUND

Approximately 5% of breast cancer patients are associated with germline mutations in the BRCA1/2 genes (3% in the BRCA1 gene and 2% in the BRCA2 gene). Most breast cancers caused by BRCA1 mutations are triple-negative breast cancers (70%), while BRCA2 mutations are more likely to cause estrogen receptor-positive breast cancers (70%). The BRCA1/2 genes are tumor suppressor genes and play an important role in DNA damage repair, normal cell growth, etc. Mutations in the genes can inhibit the normal repair ability after DNA damage and cause homologous recombination deficiency (HRD), that is, loss of BRCA function or mutation or loss of function in other homologous recombination-related genes, making repair of DNA double-strand breaks impossible through homologous recombination repair (HRR), eventually leading to cancer. Poly (ADP-ribose) polymerase (PARP) is a DNA repair enzyme that plays a key role in the DNA repair pathway. PARP is activated by DNA damage and breakage. As a molecular sensor of DNA damage, it has the function of identifying and binding to the DNA break location, thereby activating and catalyzing the polyADP ribosylation of the receptor protein and participating in the DNA repair process. PARP plays a key role in the process of DNA single-strand base excision and repair. In HRD tumor cells, DNA double-strand breaks cannot be repaired, and PARP inhibitors block the single-strand repair, resulting in a “synthetic lethal” effect and leading to tumor cell death. PARP inhibitors have a “trapping” effect on the PARP protein, causing the PARP protein that binds to damaged DNA to be trapped on the DNA, directly causing other DNA repair proteins to be unable to bind, eventually leading to cell death. At present, several PARP inhibitors have been successfully developed, such as olaparib, rucaparib and niraparib. However, adverse reactions limit their ability to be used in combination with chemotherapy drugs. This may be related to the lack of selectivity of marketed PARP inhibitors against the PARP family. The side effects include intestinal toxicity caused by tankyrase inhibition and hematological toxicity caused by PARP-2 inhibition. Therefore, it is of great clinical significance to develop highly selective PARP-1 inhibitors and reduce the toxic and side effects associated with non-selective PARP inhibitors.

When used for treating humans, it is important that a crystalline form of a therapeutic agent, like N-cyclopropyl-5-(4-((7-ethyl-6-oxo-5,6-dihydro-1,5-naphthyridin-3-yl)methyl)piperazin-1-yl)pyridine carboxamide retains its polymorphic and chemical stability, solubility, and other physicochemical properties over time and among various manufacturing batches of the agent. If the physicochemical properties vary over time and among batches, the administration of a therapeutically effective dose becomes problematic and may lead to toxic side effects or to ineffective therapy, particularly when a given polymorph decomposes prior to use, to a less active, inactive, or toxic compound. Therefore, it is very important to choose a crystal form that is stable, is manufactured reproducibly, and has physicochemical properties favorable for its use as a therapeutic agent.

### SUMMARY

The present invention relates to a compound N-cyclopropyl-5-(4-((7-ethyl-6-oxo-5,6-dihydro-1,5-naphthyridin-3-yl)methyl)piperazin-1-yl)pyridine carboxamide as shown in formula (I), a crystal form comprising the compound and a preparation method therefor, and the use thereof in a pharmaceutical composition and in medicine. The compound provided in the present invention has high selectivity, good activity, and low toxic and side effects, and a plurality of crystalline substances have excellent characteristics of high purity, good solubility, stable physical and chemical properties, resistance to high temperature, high humidity and strong illumination, low hygroscopicity, etc.

A crystalline substance of a compound as shown in formula (I):

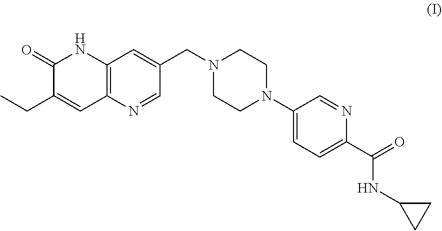 [SMILES: CCc1cc2ncc(CN3CCN(c4ccc(C(=O)NC5CC5)nc4)CC3)cc2[nH]c1=O]

In some particular embodiments, the crystalline substance is of crystal form B having an X-ray powder diffraction pattern with characteristic diffraction peaks at the following 2θ positions by using Cu-Kα radiation: 19.31°±0.2°, 20.37°±0.2°, 22.23°±0.2°, 22.90°±0.2°, 23.70°±0.2°, and 27.18°±0.2°.

In some particular embodiments, the crystalline substance is of crystal form B having an X-ray powder diffraction pattern with characteristic diffraction peaks at the following 2θ positions by using Cu-Kα radiation: 11.12°±0.2°, 15.96°±0.2°, 16.93°±0.2°, 19.31°±0.2°, 20.37°±0.2°, 22.23°±0.2°, 22.90°±0.2°, 23.70°±0.2°, 25.45°±0.2°, 26.52°±0.2°, 27.18°±0.2°, 29.14°±0.2°, and 32.78°±0.2°.

In some particular embodiments, the crystalline substance is of crystal form B having an X-ray powder diffraction pattern substantially as shown in FIG. 3 by using Cu-Kα radiation.

In some particular embodiments, the crystal form B has a differential scanning calorimetry curve and a thermogravimetric analysis curve as shown in FIG. 1 and FIG. 2, respectively.

In some particular embodiments, the crystalline substance is of crystal form E having an X-ray powder diffraction pattern with characteristic diffraction peaks at the following 2θ positions by using Cu-Kα radiation: 10.76°±0.2°, 15.03°±0.2°, 17.79°±0.2°, and 19.28°±0.2°.

In some particular embodiments, the crystalline substance is of crystal form E having an X-ray powder diffraction pattern with characteristic diffraction peaks at the following 2θ positions by using Cu-Kα radiation: 3.63°±0.2°, 7.18°±0.2°, 10.76°±0.2°, 15.03°±0.2°, 17.47°±0.2°, 17.79°±0.2°, 19.28°±0.2°, 21.33°±0.2°, 23.76°±0.2°, and 27.19°±0.2°.

In some particular embodiments, the crystalline substance is of crystal form E having an X-ray powder diffraction pattern with characteristic diffraction peaks at the following 2θ positions by using Cu-Kα radiation: 3.63°±0.2°, 7.18°±0.2°, 7.80°±0.2°, 10.27°±0.2°, 10.76°±0.2°, 15.03°±0.2°, 17.27°±0.2°, 17.47°±0.2°, 17.79°±0.2°, 19.28°±0.2°, 20.09°±0.2°, 20.63°±0.2°, 21.33°±0.2°, 22.41°±0.2°, 23.76°±0.2°, 24.02°±0.2°, 25.89°±0.2°, 27.19°±0.2°, and 27.67°±0.2°.

In some particular embodiments, the crystal form E has an X-ray powder diffraction pattern substantially as shown in FIG. 6 by using Cu-Kα radiation.

In some particular embodiments, the crystal form E has a differential scanning calorimetry curve and a thermogravimetric analysis curve as shown in FIG. 4 and FIG. 5, respectively.

In some particular embodiments, the crystalline substance is of crystal form F having an X-ray powder diffraction pattern with characteristic diffraction peaks at the following 2θ positions by using Cu-Kα radiation: 9.66°±0.2°, 10.70°±0.2°, 14.24°±0.2°, 19.26°±0.2°, 21.11°±0.2°, and 22.10°±0.2°.

In some particular embodiments, the crystalline substance is of crystal form F having an X-ray powder diffraction pattern with characteristic diffraction peaks at the following 2θ positions by using Cu-Kα radiation: 9.66°±0.2°, 10.70°±0.2°, 14.24°±0.2°, 17.34°±0.2°, 19.26°±0.2°, 21.11°±0.2°, 22.10°±0.2°, and 24.77±0.2°.

In some particular embodiments, the crystalline substance is of crystal form F having an X-ray powder diffraction pattern substantially as shown in FIG. 9 by using Cu-Kα radiation.

In some particular embodiments, the crystalline substance is of crystal form F having a differential scanning calorimetry curve and a thermogravimetric analysis curve as shown in FIG. 7 and FIG. 8, respectively.

In some particular embodiments, the crystalline substance is of crystal form G having an X-ray powder diffraction pattern with characteristic diffraction peaks at the following 2θ positions by using Cu-Kα radiation: 7.81°±0.2°, 10.30°±0.2°, 11.70°±0.2°, 19.44°±0.2°, and 20.66°±0.2°.

In some particular embodiments, the crystalline substance is of crystal form G having an X-ray powder diffraction pattern with characteristic diffraction peaks at the following 2θ positions by using Cu-Kα radiation: 7.81°±0.2°, 9.47°±0.2°, 10.30°±0.2°, 11.70°±0.2°, 12.37°±0.2°, 19.44°±0.2°, 19.75°±0.2°, 20.03°±0.2°, 20.41°±0.2°, 20.66°±0.2°, 22.49°±0.2°, and 26.77°±0.2°.

In some particular embodiments, the crystalline substance is of crystal form G having an X-ray powder diffraction pattern with characteristic diffraction peaks at the following 2θ positions by using Cu-Kα radiation: 7.81°±0.2°, 9.47°±0.2°, 10.30°±0.2°, 11.70°±0.2°, 12.37°±0.2°, 17.27°±0.2°, 19.44°±0.2°, 19.75°±0.2°, 20.03°±0.2°, 20.41°±0.2°, 20.66°±0.2°, 21.18°±0.2°, 21.60°±0.2°, 22.49°±0.2°, 26.38°±0.2°, 26.77°±0.2°, 27.50°±0.2°, 28.96°±0.2°, 33.69°±0.2°, and 37.22°±0.2°.

In some particular embodiments, the crystalline substance is of crystal form G having an X-ray powder diffraction pattern substantially as shown in FIG. 12 by using Cu-Kα radiation.

In some particular embodiments, the crystalline substance is of crystal form G having a differential scanning calorimetry curve and a thermogravimetric analysis curve as shown in FIG. 10 and FIG. 11, respectively.

In some particular embodiments, the crystalline substance is of crystal form H having an X-ray powder diffraction pattern with characteristic diffraction peaks at the following 2θ positions by using Cu-Kα radiation: 5.11°±0.2°, 8.73°±0.2°, 10.39°±0.2°, 15.92°±0.2°, 16.99°±0.2°, 17.34°±0.2°, 18.07°±0.2°, 21.09°±0.2°, 23.46°±0.2°, 24.79°±0.2°, 25.49°±0.2°, and 26.33°±0.2°.

In some particular embodiments, the crystalline substance is of crystal form H having an X-ray powder diffraction pattern substantially as shown in FIG. 15 by using Cu-Kα radiation.

In some particular embodiments, the crystal form H has a differential scanning calorimetry curve and a thermogravimetric analysis curve as shown in FIG. 13 and FIG. 14, respectively.

Optionally, the present invention provides a crystal form of a hydrate of compound I, and optionally, the hydrate has a water content of 0.1-4, 0.1-3, 0.1-2 or 0.1-1; and optionally, the hydrate has a water content of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0.

The present invention further provides a pharmaceutical composition, wherein the pharmaceutical composition comprises a therapeutically effective amount of any one of the preceding crystalline substances, and a pharmaceutically acceptable carrier and/or excipient, and the therapeutically effective amount is preferably 1-600 mg calculated based on a free base. The pharmaceutical composition can be in a unit preparation form (the unit preparation is also referred to as “preparation specification”).

The present invention further provides the use of the crystalline substance or composition according to any one of the preceding solutions in the preparation of a drug for treating/preventing a PARP-mediated disease. Further, the PARP-mediated disease is a tumor.

The present invention further provides a method for treating a disease in a mammal, comprising administering to a subject a therapeutically effective amount of the crystalline substance or composition according to any one of the preceding solutions, wherein the disease is preferably a tumor, and the therapeutically effective amount is preferably 1-600 mg calculated based on a free base. In some embodiments, the mammal mentioned in the present invention includes human.

The “effective amount” or “therapeutically effective amount” as described in the present application refers to administration of a sufficient amount of the crystalline substance disclosed in the present application that will alleviate to some extent one or more symptoms of the diseases or conditions being treated. In some embodiments, the outcome is the reduction and/or remission of signs, symptoms or causes of the disease, or any other desired change in the biological system. For example, an “effective amount” in terms of the therapeutic use is an amount of the composition comprising the crystalline substance disclosed in the present application that is required to provide clinically significant reduction of the symptoms of the disease. Examples of the therapeutically effective amount (calculated based on a free base) include, but are not limited to 1-600 mg, 1-500 mg, 1-400 mg, 1-300 mg, 1-250 mg, 1-200 mg, 1-150 mg, 1-125 mg, 1-100 mg, 1-80 mg, 1-60 mg, 1-50 mg, 1-40 mg, 1-25 mg, 1-20 mg, 5-300 mg, 5-250 mg, 5-200 mg, 5-150 mg, 5-125 mg, 5-100 mg, 5-90 mg, 5-70 mg, 5-80 mg, 5-60 mg, 5-50 mg, 5-40 mg, 5-30 mg, 5-25 mg, 5-20 mg, 10-600 mg, 10-500 mg, 10-450 mg, 10-400 mg, 10-300 mg, 10-250 mg, 10-200 mg, 10-150 mg, 10-125 mg, 10-100 mg, 10-90 mg, 10-80 mg, 10-70 mg, 10-60 mg, 10-50 mg, 10-40 mg, 10-30 mg, 10-20 mg; 20-600 mg, 20-500 mg, 20-400 mg, 20-350 mg, 20-300 mg, 20-250 mg, 20-200 mg, 20-150 mg, 20-125 mg, 20-100 mg, 20-90 mg, 20-80 mg, 20-70 mg, 20-60 mg, 20-50 mg, 20-40 mg, 20-30 mg; 50-600 mg, 50-500 mg, 50-400 mg, 50-300 mg, 50-250 mg, 50-200 mg, 50-150 mg, 50-125 mg, 50-100 mg; 100-600 mg, 100-500 mg, 100-400 mg, 100-300 mg, 100-250 mg and 100-200 mg.

In some embodiments, the pharmaceutical composition or preparation of the present invention contains the above-described therapeutically effective amount of the crystalline substance of the present invention.

The present invention relates to a pharmaceutical composition or pharmaceutical preparation, comprising a therapeutically effective amount of the crystalline substance according to the present invention, and a carrier and/or an excipient. The pharmaceutical composition can be in a unit preparation form (the amount of the active drug in the unit preparation is also referred to as the “preparation specification”). In some embodiments, the pharmaceutical composition (calculated based on a free base) includes, but is not limited to 1 mg, 1.25 mg, 2.5 mg, 5 mg, 10 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 275 mg, 300 mg, 325 mg, 350 mg, 375 mg, 400 mg, 425 mg, 450 mg, 475 mg, 500 mg, 525 mg, 550 mg, 575 mg and 600 mg.

A method for treating a disease in a mammal, comprising administering to a subject a therapeutically effective amount of the crystalline substance according to the present invention, and a pharmaceutically acceptable carrier and/or excipient, wherein the therapeutically effective amount is preferably 1-600 mg calculated based on a free base, and the disease is preferably a tumor, especially a brain tumor.

A method for treating a disease in a mammal, comprising administering to a subject a drug, i.e., the crystalline substance according to the present invention, and a pharmaceutically acceptable carrier and/or excipient at a daily dose of 1-600 mg/day calculated based on a free base, wherein the daily dose may be a single dose or divided doses; in some embodiments, the daily dose includes, but is not limited to, 10-600 mg/day, 20-600 mg/day, 25-600 mg/day, 50-600 mg/day, 75-600 mg/day, 100-600 mg/day, 200-600 mg/day, 10-600 mg/day, 20-600 mg/day, 25-600 mg/day, 50-600 mg/day, 75-600 mg/day, 100-600 mg/day, 200-600 mg/day, 25-600 mg/day, 50-600 mg/day, 100-600 mg/day, 200-600 mg/day, 25-400 mg/day, 50-400 mg/day, 100-400 mg/day and 200-400 mg/day; and in some embodiments, the daily dose includes, but is not limited to, 1 mg/day, 5 mg/day, 10 mg/day, 20 mg/day, 25 mg/day, 50 mg/day, 75 mg/day, 100 mg/day, 125 mg/day, 150 mg/day, 200 mg/day, 400 mg/day and 600 mg/day.

The present invention relates to a kit, wherein the kit may comprise a crystalline substance in the form of a single dose or multiple doses and comprises the crystalline substance according to the present invention, and the amount of the crystalline substance according to the present invention is identical to the amount of same in the above-described pharmaceutical composition.

In the present invention, the amount of the crystalline substance according to the present invention is calculated in the form of a free base in each case.

The term “preparation specification” refers to the weight of the active drug contained in each vial, tablet or other unit preparation.

The crystalline substance according to the present invention is present in an amount accounting for about 5 wt % to about 100 wt % of the bulk drug; in certain embodiments, in an amount accounting for about 10 wt % to about 100 wt % of the bulk drug; in certain embodiments, in an amount accounting for about 15 wt % to about 100 wt % of the bulk drug; in certain embodiments, in an amount accounting for about 20 wt % to about 100 wt % of the bulk drug; in certain embodiments, in an amount accounting for about 25 wt % to about 100 wt % of the bulk drug; in certain embodiments, in an amount accounting for about 30 wt % to about 100 wt % of the bulk drug; in certain embodiments, in an amount accounting for about 35 wt % to about 100 wt % of the bulk drug; in certain embodiments, in an amount accounting for about 40 wt % to about 100 wt % of the bulk drug; in certain embodiments, in an amount accounting for about 45 wt % to about 100 wt % of the bulk drug; in certain embodiments, in an amount accounting for about 50 wt % to about 100 wt % of the bulk drug; in certain embodiments, in an amount accounting for about 55 wt % to about 100 wt % of the bulk drug; in certain embodiments, in an amount accounting for about 60 wt % to about 100 wt % of the bulk drug; in certain embodiments, in an amount accounting for about 65 wt % to about 100 wt % of the bulk drug; in certain embodiments, in an amount accounting for about 70 wt % to about 100 wt % of the bulk drug; in certain embodiments, in an amount accounting for about 75 wt % to about 100 wt % of the bulk drug; in certain embodiments, in an amount accounting for about 80 wt % to about 100 wt % of the bulk drug; in certain embodiments, in an amount accounting for about 85 wt % to about 100 wt % of the bulk drug; in certain embodiments, in an amount accounting for about 90 wt % to about 100 wt % of the bulk drug; in certain embodiments, in an amount accounting for about 95 wt % to about 100 wt % of the bulk drug; in certain embodiments, in an amount accounting for about 98 wt % to about 100 wt % of the bulk drug; in certain embodiments, in an amount accounting for about 99 wt % to about 100 wt % of the bulk drug; and in certain embodiments, substantially all of the bulk drug are substantially pure crystals.

The crystalline substance of the present invention can be prepared according to the following preparation method:
1. Volatilization experiment: a clear solution of a sample is exposed to an atmosphere at various temperatures until the solvent is volatilized and removed.
2. Crystal slurrying experiment: a supersaturated solution of a sample (containing an undissolved solid) is stirred in different solvent systems at a certain temperature.
3. Anti-solvent experiment: a sample is dissolved in a good solvent, an anti-solvent (a poor solvent) is added to precipitate a solid, followed by brief stirring and immediate filtration.
4. Cooling crystallization experiment: a certain amount of samples are dissolved in a corresponding solvent at a high temperature, and the mixture is directly stirred at room temperature or a low temperature for crystallization.
5. Polymer template experiment: various polymer materials are added to a clear solution of a sample, and the resulting solution is exposed to an atmosphere at room temperature until the solvent is volatilized and removed.
6. Thermal method experiment: a sample is treated according to a certain thermal method under crystallization conditions and cooled to room temperature.
7. Water vapor diffusion experiment: a sample is left in a certain humidity environment at room temperature.

The good and poor solvents according to the present invention are relative; and in a pair of solvents, the one with a higher solubility is a good solvent and the one with a lower solubility is a poor solvent. The solvents used in the above-described preparation methods may be a single solvent or a combination of two or more solvents, if not specified.

Other patterns substantially the same as the X-ray powder diffraction pattern, DSC pattern or TGA pattern disclosed in the present invention also fall within the scope of the present invention.

Unless stated to the contrary, the terms used in the description and the claims have the following meanings.

The “IC₅₀” refers to the half maximal inhibitory concentration, i.e., a concentration where half of the maximum inhibitory effect is achieved.

The “ether solvent” refers to a chain compound or a cyclic compound containing an ether bond —O— and having 2 to 10 carbon atoms, and the specific examples thereof include, but are not limited to: tetrahydrofuran, diethyl ether, propylene glycol methyl ether, methyl tert-butyl ether, isopropyl ether or 1,4-dioxane.

The “alcohol solvent” refers to a group derived from “C₁₋₆ alkyl” on which one or more hydrogen atoms are substituted with one or more “hydroxyl groups”, wherein the “hydroxyl group” and “C₁₋₆ alkyl” are as defined above, and the specific examples thereof include, but are not limited to: methanol, ethanol, isopropanol, n-propanol, isopentanol or trifluorocthanol.

The “ester solvent” refers to a combination of a lower organic acid containing 1-4 carbon atoms and a lower alcohol containing 1-6 carbon atoms, and the specific examples thereof include, but are not limited to: ethyl acetate, isopropyl acetate or butyl acetate.

The “ketone solvent” refers to a compound in which a carbonyl group (—C(O)—) is connected to two hydrocarbon groups. According to the difference of hydrocarbon groups in molecules, ketones can be divided into aliphatic ketone, alicyclic ketone, aromatic ketone, saturated ketone and unsaturated ketone, and the specific examples thereof include, but are not limited to: acetone, acetophenone and 4-methyl-2-pentanone.

The “nitrile solvent” refers to a group derived from “C₁₋₆ alkyl” on which one or more hydrogen atoms are substituted with one or more “cyano groups”, wherein the “cyano group” and “C₁₋₆ alkyl” are as defined above, and the specific examples thereof include, but are not limited to: acetonitrile or propionitrile.

The “halogenated hydrocarbon solvent” refers to a group derived from “C₁₋₆ alkyl” on which one or more hydrogen atoms are substituted with one or more “halogen atoms”, wherein the “halogen atom” and “C₁₋₆ alkyl” are as defined above, and the specific examples thereof include, but are not limited to: dichloromethane, 1,2-dichlorocthane, chloroform or carbon tetrachloride.

As used in the present invention, “the crystal of the present invention”, “the crystal form of the present invention”, “the crystalline substance of the present invention” and the like can be used interchangeably.

The “room temperature” of the present invention generally refers to 4° C. to 30° C., preferably 20° C.±5° C.

The structure of the crystal form of the present invention can be analyzed by using various analytical techniques known to those skilled in the art, including but not limited to X-ray powder diffraction (XRD), differential scanning calorimetry (DSC) and/or thermogravimetric analysis (TGA), also known as thermogravimetry (TG).

The “2θ or 2θ angle” according to the present invention refers to the position of the peak expressed in degrees (°) and set based on the X-ray diffraction experiment and is typically the unit of the abscissa in the diffraction pattern. If the reflection beam is diffracted when the incident beam forms an angle θ with a lattice plane, the experimental setup requires that the reflection beam be recorded at an angle 2θ. It should be understood that a particular 2θ value for a particular crystal form referred to herein is intended to mean the 2θ value (expressed in degrees) measured using the X-ray diffraction experimental conditions described herein, and the error range of the 2θ may be ±0.3, +0.5 or ±0.1.

It can be understood that the numerical values described and claimed in the present invention are approximate values. Changes in values may be attributed to device calibration, device errors, crystal purity, crystal size, sample size and other factors.

It can be understood that the crystal forms of the present invention are not limited to the characteristic patterns such as XRD, DSC and TGA which are completely identical to those described in the drawings disclosed in the present invention, and any crystal form having a characteristic pattern which is essentially or substantially the same as those described in the drawings falls within the scope of the present invention.

It can be understood that, as is well known in the field of differential scanning calorimetry (DSC), a melting peak height of a DSC curve depends on many factors related to sample preparation and geometric shapes of instruments, and a peak position is relatively insensitive to experiment details. Therefore, in some embodiments, the crystallized compounds of the present invention have DSC patterns showing characteristic peak positions, which have essentially the same properties as the DSC patterns provided in the drawings of the present invention, with an error tolerance of measured values within ±5° C., which is generally required to be within ±3° C.

The term “carrier” refers to: a system that does not cause significant irritation to the organism and does not eliminate the biological activity and characteristics of the administered compound and can change the way the drug enters the human body and the distribution of the drug in the body, control the release rate of the drug and delivery the drug to targeted organs. Non-limiting examples of the carrier include microcapsule, microsphere, nanoparticle, liposome, etc.

The term “excipient” refers to: a substance that is not a therapeutic agent per se, but used as a diluent, adjuvant, adhesive and/or vehicle for addition to a pharmaceutical composition, thereby improving the disposal or storage properties thereof, or allowing to or promoting the formation of a compound or a pharmaceutical composition into a unit dosage form for administration. As is known to those skilled in the art, pharmaceutically acceptable excipients can provide various functions and can be described as a wetting agent, a buffer, a suspending agent, a lubricant, an emulsifier, a disintegrating agent, an absorbent, a preservative, a surfactant, a colorant, a flavoring agent, and a sweetening agent. Examples of pharmaceutically acceptable excipients include, but are not limited to: (1) sugars, such as lactose, glucose, and sucrose; (2) starch, such as corn starch and potato starch; (3) cellulose and derivatives thereof, such as sodium carboxymethyl cellulose, ethyl cellulose, cellulose acetate, hydroxypropyl methylcellulose, hydroxypropyl cellulose, microcrystalline cellulose, and croscarmellose (such as croscarmellose sodium); (4) tragacanth powder; (5) malt; (6) gelatin; (7) talc; (8) excipients, such as cocoa butter or suppository wax; (9) oils, such as peanut oil, cottonseed oil, safflower oil, sesame oil, olive oil, corn oil, and soybean oil; (10)diols, such as propylene glycol; (11) polyols, such as glycerol, sorbitol, mannitol, and polyethylene glycol; (12) esters, such as ethyl oleate and ethyl laurate; (13) agar; (14) buffers, such as magnesium hydroxide and aluminum hydroxide; (15) alginic acid; (16) pyrogen-free water; (17) isotonic saline; (18) Ringer's solution; (19) ethanol; (20) pH buffer solution; (21) polyester, polycarbonate and/or polyanhydride; and (22) other non-toxic compatible substances used in a pharmaceutical preparation.

### BRIEF DESCRIPTION OF THE DRAWINGS

FIG. 1 shows the differential scanning calorimetry curve pattern of the crystal form B of the compound as shown in formula (I).

FIG. 2 shows the thermogravimetric analysis pattern of the crystal form B of the compound as shown in formula (I).

FIG. 3 shows the X-ray powder diffraction pattern of the crystal form B of the compound as shown in formula (I).

FIG. 4 shows the differential scanning calorimetry curve pattern of the crystal form E of the compound as shown in formula (I).

FIG. 5 shows the thermogravimetric analysis pattern of the crystal form E of the compound as shown in formula (I).

FIG. 6 shows the X-ray powder diffraction pattern of the crystal form E of the compound as shown in formula (I).

FIG. 7 shows the differential scanning calorimetry curve pattern of the crystal form F of the compound as shown in formula (I).

FIG. 8 shows the thermogravimetric analysis pattern of the crystal form F of the compound as shown in formula (I).

FIG. 9 shows the X-ray powder diffraction pattern of the crystal form F of the compound as shown in formula (I).

FIG. 10 shows the differential scanning calorimetry curve pattern of the crystal form G of the compound as shown in formula (I).

FIG. 11 shows the thermogravimetric analysis pattern of the crystal form G of the compound as shown in formula (I).

FIG. 12 shows the X-ray powder diffraction pattern of the crystal form G of the compound as shown in formula (I).

FIG. 13 shows the differential scanning calorimetry curve pattern of the crystal form H of the compound as shown in formula (I).

FIG. 14 shows the thermogravimetric analysis pattern of the crystal form H of the compound as shown in formula (I).

FIG. 15 shows the X-ray powder diffraction pattern of the crystal form H of the compound as shown in formula (I).

FIG. 16 shows the tumor growth curve of the mouse MDA-MB-436 subcutaneous in vivo transplanted tumor model.

FIG. 17 shows the animal body weight change curve of the mouse MDA-MB-436 subcutaneous in vivo transplanted tumor model.

### DETAILED DESCRIPTION

The technical solutions of the present invention will be described in detail below in conjunction with the drawings and examples, but the scope of protection of the present invention includes but is not limited thereto.

The structures of the compounds are determined by nuclear magnetic resonance (NMR) or (and) mass spectrometry (MS). The NMR shift (8) is given in the unit of 10⁻⁶ (ppm). NMR is determined with (Bruker Avance III 400 and Bruker Avance 300) nuclear magnetic resonance instrument; the solvents for determination are deuterated dimethyl sulfoxide (DMSO-d₆), deuterated chloroform (CDCl₃) and deuterated methanol (CD₃OD); and the internal standard is tetramethylsilane (TMS).

MS is determined with Agilent 6120B (ESI) and Agilent 6120B (APCI).

HPLC is determined with LC-20AT (Shimadzu) high pressure liquid chromatograph (Kromasil 100-5-C18, 4.6 mm×250 mm).

XRD is determined with an X-ray powder diffractometer, Bruker D8 Advance Diffractometer. X-ray powder diffraction test is performed according to the following method.

TABLE 1
Test parameters of XRD

| | Empyrean (variable | Empyrean or X′ Pert3 |
|---|---|---|
| Model | temperature mode) | (reflection mode) |
| X ray | Cu, Kα, | Cu, Kα, |
| | Kα1 (Å): 1.540598, | Kα1 (Å): 1.540598, |
| | Kα2 (Å): 1.544426 | Kα2 (Å): 1.544426 |
| | Kα2/Kα1 intensity | Kα2/Kα1 intensity |
| | ratio: 0.50 | ratio: 0.50 |
| Settings of | 45 kV, 40 mA | 45 kV, 40 mA |
| X-ray tube | | |
| Divergence slit | 1/8° | 1/8° |
| Scan mode | Continuous | Continuous |
| Scan range | 3-40 | 3-40 |
| (°2Theta) | | |
| Scanning time | 17.8 | 46.7 |
| per step (s) | | |
| Step length of | 0.0167 | 0.0263 |
| scanning | | |
| (°2Theta) | | |
| Test time | About 10 min | About 5 min |

TGA and DSC patterns are obtained on a TA 5500 thermal gravimetric analyzer and a TA 2500 differential scanning calorimeter, respectively, and test parameters are as shown in the table below.

TABLE 2
Test parameters of DSC and TGA

| Parameter | TGA | DSC |
|---|---|---|
| Method | Linear | Linear |
| | temperature rise | temperature rise |
| Sample tray | Aluminum tray, | Aluminum tray, |
| | open | capped/uncapped |
| Temperature range | Room temperature - set | 25° C. - set |
| | final temperature | final temperature |
| Scanning rate | 10 | 10 |
| (° C./min) | | |
| Protective gas | Nitrogen gas | Nitrogen gas |

The known starting materials of the present invention can be synthesized by or according to methods known in the art, or can be purchased from Titan Technology Co., Ltd., Energy Chemical Co., Ltd., Shanghai Demo Co., Ltd., Chengdu Kelong Chemical Co., Ltd., Accela ChemBio Co., Ltd., J&K Scientific Co., Ltd. and other companies.

Unless otherwise specified in the examples, a solution refers to an aqueous solution.

Unless otherwise specified in the examples, the room temperature is 20° C. to 30° C.

The implementation process and beneficial effects of the present invention are described in detail below through specific examples, which are intended to help readers better understand the essence and characteristics of the present invention, and are not intended to limit the scope of implementation of the present invention.
DMAc: N,N-dimethylacetamide
NMP: N-methylpyrrolidone
MTBE: Methyl tert-butyl ether

### Example 1: Preparation of Compound I

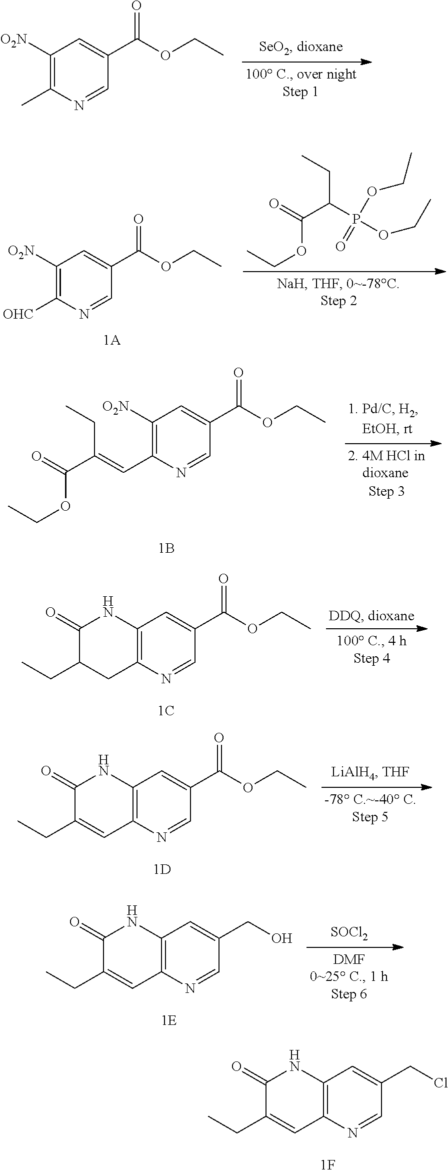

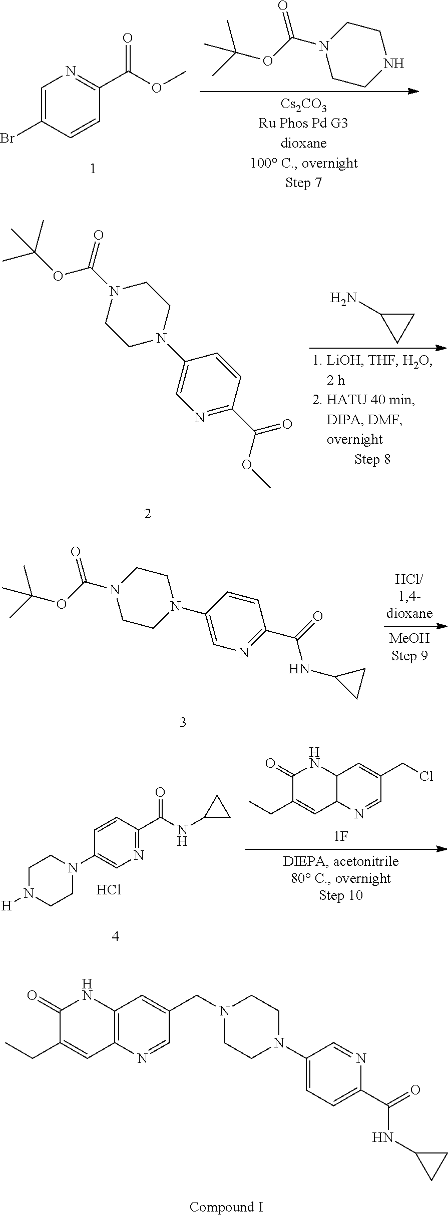

### Step 1:

Ethyl 6-methyl-5-nitronicotinate (10 g, 47.6 mmol) and selenium dioxide (21.14 g, 190.5 mmol) were dissolved in 1,4-dioxane (100 ml), and the mixture was refluxed overnight at 100° C. After the reaction was completed, the reaction liquid was filtered through a funnel lined with diatomaceous earth, the diatomaceous earth was washed with ethyl acetate, the filtrate was concentrated, and the resulting residue was separated and purified by silica gel column chromatography (eluent: ethyl acetate:petroleum ether (v/v)=0%-40%), to afford compound 1A (10.104 g, 94.7%) as a yellow oil.

LCMS (ESI) m/z=225.1 [M+1]⁺

### Step 2:

Sodium hydride (2.695 g, 112.3 mmol) was dissolved in anhydrous tetrahydrofuran (100 ml) and stirred at 0° C., and triethyl 2-phosphonobutyrate (28.3 g, 112.3 mmol) was added dropwise. After the dropwise addition was completed, the mixture was stirred at 0° C. for 20 min, warmed to 40° C. and stirred for 10 min, and then transferred to a dry ice ethanol bath. Compound 1A (10.48 g, 46.8 mmol) was dissolved in anhydrous tetrahydrofuran (100 ml) and added dropwise to a reaction flask. The mixture was kept in the dry ice ethanol bath and stirred for 1 h. After the reaction was completed, the reaction liquid was quenched with saturated ammonium chloride solution (100 ml) and extracted with ethyl acetate (200 ml). The organic phase was separated, and the aqueous phase was extracted with ethyl acetate (200 ml×2). The organic phases were combined, dried over anhydrous sodium sulfate and concentrated, and the resulting residue was purified by silica gel column chromatography (eluent: ethyl acetate:petroleum ether (v/v)=0-10%), to afford compound 1B (11.57 g, 76.8%) as a yellow oil, a mixture of two isomers.

LC-MS (ESI) m/z=323.1 [M+1]⁺

### Step 3:

Compound 1B (11.57 g, 35.9 mmol) was dissolved in ethanol (50 ml), and 10% palladium on carbon catalyst (1 g) was added. The mixture was subjected to hydrogen replacement three times, stirred overnight at room temperature, and filtered through a funnel lined with diatomaceous earth, and the diatomaceous earth was washed with anhydrous ethanol. The filtrate was concentrated, to the resulting residue was added a solution of hydrogen chloride in dioxane (60 ml, 4M), and the mixture was stirred at room temperature for 1 h and concentrated. To the resulting residue was added ethyl acetate (50 ml), the mixture was stirred and filtered, and the filter cake was washed with ethyl acetate and dried, to afford compound 1C (4.28 g, 42.0%) as a white solid.

¹H NMR (400 MHZ, DMSO-d₆) δ 10.39 (s, 1H), 8.62 (d, 1H), 7.75 (s, 1H), 4.38-4.29 (m, 2H), 3.24 (dd, 1H), 2.97 (dd, 1H), 2.62-2.53 (m, 1H), 1.83-1.64 (m, 1H), 1.55-1.35 (m, 1H), 1.33 (dd, 3H), 0.94 (t, 3H).

### Step 4:

Compound 1C (4.28 g, 17.3 mmol) and 2,3-dichloro-5,6-dicyanobenzoquinone (4.309 g, 19.0 mmol) were dissolved in dioxane (86 ml), and the mixture was reacted at 100° C. for 3.5 h at reflux. After the reaction was completed, saturated sodium bicarbonate solution (40 ml) and ethyl acetate (120 ml) were added. The organic phase was separated, and the aqueous phase was extracted with ethyl acetate (120 ml×2). The organic phases were combined, dried over anhydrous sodium sulfate and concentrated, and the resulting residue was purified by silica gel column chromatography (eluent: ethyl acetate:petroleum ether=0-50%), to afford compound 1D (3.375 g, 79.5%) as a pale yellow solid.

LC-MS (ESI) m/z=247.1 [M+1]⁺

### Step 5:

Compound 1D (3.375 g, 13.72 mmol) was dissolved in anhydrous tetrahydrofuran (150 ml), and the mixture was stirred at −78° C. Lithium aluminum hydride (1.564 g, 41.16 mmol) was added portionwise, and the mixture was stirred at −78° C. for 20 min and then warmed to −40° C. and stirred for 20 min. After the reaction was completed, 1M hydrochloric acid was added to adjust the system to a neutral pH, the solvent was removed by distillation under reduced pressure, to the resulting residue was added methanol/dichloromethane (1:10, 100 ml) for dissolution, and the mixture was subjected to ultrasonic vibration for 10 min and filtered. The filtrate was collected, the filter cake was redissolved in methanol/dichloromethane (1:10, 100 ml), and this process was repeated 8 times. The filtrate was combined and concentrated, to afford compound 1E (2.8 g, 100%) as a pale yellow solid.

¹H NMR (400 MHZ, DMSO) δ 11.86 (s, 1H), 8.37 (d, 1H), 7.72 (d, 1H), 7.62 (d, 1H), 5.44 (t, 1H), 4.61 (d, 2H), 2.57-2.51 (m, 2H), 1.18 (t, 3H).

### Step 6:

1E (100 mg, 0.49 mmol) was added to dichloromethane (2.5 mL), DMF (1 mL) was added to assist dissolution, thionyl chloride (350 mg, 2.94 mmol) was added dropwise at 0° C., and the mixture was reacted at room temperature for 1 hour. Upon complete depletion of raw materials monitored by LCMS, a product was generated, and the system was directly spun to dryness, to afford compound 1F (109 mg, crude) which was used in the next reaction.

LC-MS (ESI): m/z=223.1, 225.1 [M+H]⁺

### Step 7:

Methyl 5-bromopyridine-2-carboxylate (2.16 g, 10 mmol) and N-Boc-piperazine (2.03 g, 11 mmol) were dissolved in 1,4-dioxane (100 mL), Cs2CO3 (6.5 g, 20 mmol) and RuPhos-Pd-G3 (253 mg, 0.3 mmol) were added, and the mixture was reacted overnight at 100° C. under nitrogen protection. After the reaction was completed as monitored by LCMS, the reaction was terminated, cooled to room temperature, and filtered. The filtrate was collected, and the filter residue was washed with ethyl acetate (20 mL×3). The filtrate was concentrated, a small amount of anhydrous ethanol was added, and the mixture was heated and dissolved. A large amount of petroleum ether was then added, and the mixture was cooled to collect the precipitated crystals, to afford compound 2 (2.37 g, 73.4%) as a pale yellow solid.

LC-MS (ESI): m/z=321.1 [M+H]⁺.

### Step 8:

Compound 2 (400 mg, 1.24 mmol) was dissolved in THF (10 mL) and H2O (1 mL), LiOH (30 mg, 1.24 mmol) was added, and the mixture was stirred and reacted at room temperature for 2 h. The solvent was removed by distillation under reduced pressure, water was added for dilution, and the mixture was extracted with ethyl acetate (20 mL×3). The organic phases were combined, dried over anhydrous Na2SO4, filtered, and spun to dryness. To the resulting solid was added DMF (10 mL), HATU (565 mg, 1.49 mmol) was added under stirring, and the mixture was stirred at room temperature. After the solid was completely dissolved, DIEPA (2 mL) was added, excess cyclopropylamine was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE: EA (v/v)=1:0-1:1), to afford compound 3 (309 mg, 71.5%) as a pale yellow solid.

LC-MS (ESI): m/z=347.2 [M+H]⁺.

### Step 9:3

(309 mg, 0.89 mmol) was dissolved in methanol (5 mL), and a solution of hydrogen chloride in dioxane (5 mL, 4 M) was added. The mixture was reacted at room temperature for two hours and spun to dryness to obtain compound 4 (200 mg, crude).

LC-MS (ESI): m/z=247.1 [M+H]⁺.

### Step 10:

1F (100 mg, 0.44 mmol) and compound 4 (200 mg, 0.81 mmol) were dissolved in anhydrous acetonitrile (10 mL), potassium iodide (8 mg, 0.05 mmol) and DIPEA (0.5 mL) were added, and the mixture was subjected to nitrogen replacement and reacted at 80° C. for 8 hours. Upon complete depletion of raw materials monitored by LCMS, a product was generated, and the system was concentrated, saturated sodium bicarbonate solution (20 mL) was added, and the mixture was extracted with a mixed solution of DCM: MeOH (v/v)=10:1 (10 mL×3). The organic phases were combined, dried over anhydrous sodium sulfate, concentrated, and passed through column (DCM: MeOH (v/v)=1:0-10:1), to afford compound I (76 mg, 38.1%).

¹H NMR (400 MHZ, DMSO-d₆) δ 11.84 (s, 1H), 8.40 (d, 1H), 8.32 (d, 1H), 8.23 (d, 1H), 7.83 (d, 1H), 7.75 (s, 1H), 7.63 (d, 1H), 7.39 (dd, 1H), 3.65 (s, 2H), 3.35-3.31 (m, 4H, overlapped with solvent DMSO peak), 2.90-2.80 (m, 1H), 2.59-2.52 (m, 6H, overlapped with solvent DMSO peak), 1.19 (t, 3H), 0.66 (dd, 2H), 0.63 (q, 2H).

LC-MS (ESI): m/z=433.2 [M+H]⁺.

### Preparation of Crystal Form

### Example 2: Preparation of Crystal Form B

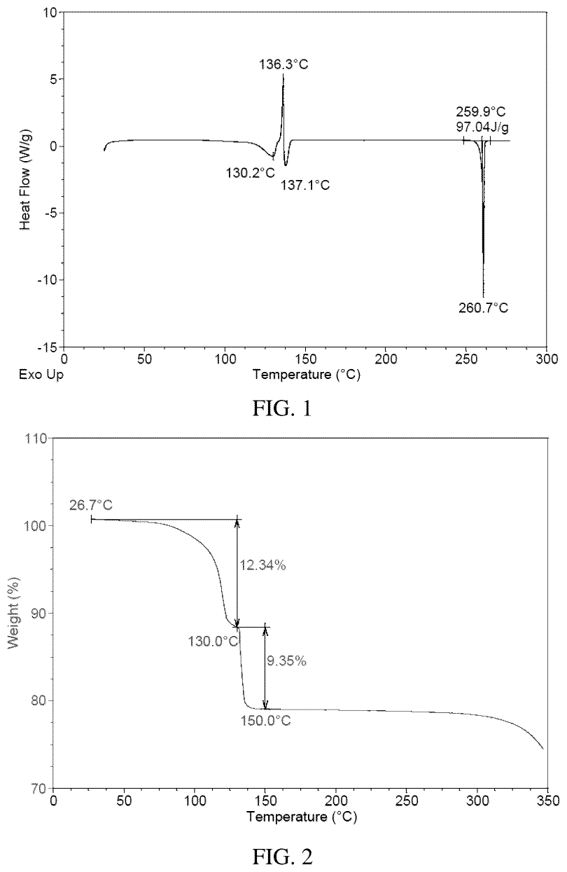

To the compound of formula I (50 mg), CHCl₃ (1 ml) was added, and the mixture was suspended at room temperature and stirred for 3 days, filtered and dried, to afford crystal form B. The differential scanning calorimetry curve pattern, thermogravimetric analysis pattern and X-ray powder diffraction pattern (XRD) of the crystal form B of the compound I were as shown in FIGS. 1-3. Specific peak values were as shown in Table 3.

TABLE 3

| Angle | d Value | Rel. |
|---|---|---|
| [°2Th.] | [Å] | Intensity [%] |
| 11.12 | 7.96 | 18.48 |
| 15.96 | 5.55 | 5.11 |
| 16.93 | 5.24 | 22.12 |
| 19.31 | 4.60 | 67.94 |
| 20.37 | 4.36 | 39.85 |
| 22.23 | 4.00 | 37.46 |
| 22.90 | 3.88 | 100.00 |
| 23.70 | 3.75 | 37.81 |
| 25.45 | 3.50 | 8.19 |
| 26.52 | 3.36 | 17.32 |
| 27.18 | 3.28 | 40.62 |
| 29.14 | 3.06 | 9.87 |
| 32.78 | 2.73 | 3.75 |

### Example 3: Preparation of Crystal Form E

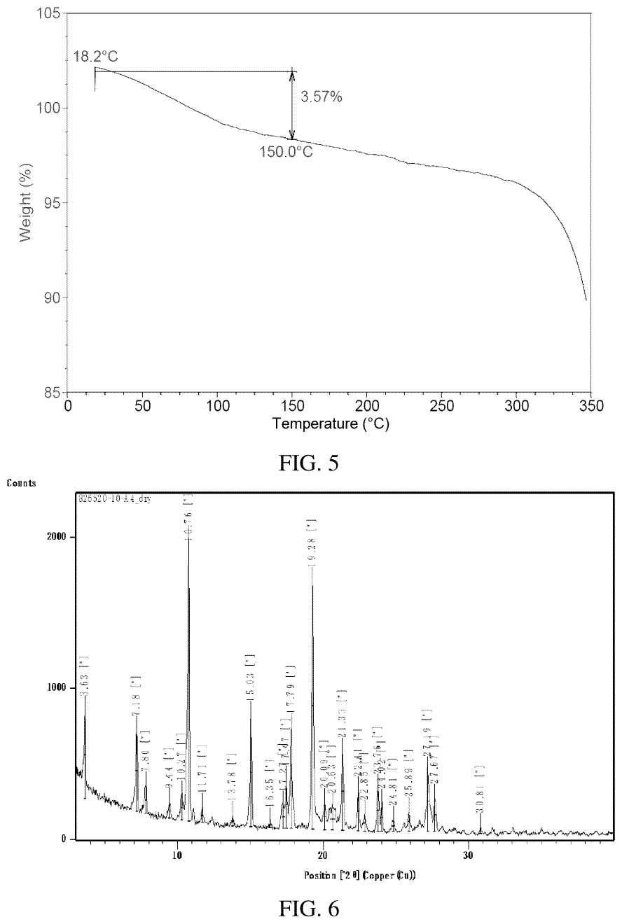

To the compound of formula I (50 mg), DMAc (1 ml) was added, and temperature was slowly cooled from room temperature. The sample was clarified after slowly cooling to 5° C., transferred and placed at −20° C. for 11 days to precipitate the solid, and exposed to an atmosphere at room temperature for drying for 1 day, and then transferred to a vacuum drying oven and dried under vacuum at room temperature for 2 days, to afford crystal form E. The differential scanning calorimetry curve pattern, thermogravimetric analysis pattern and X-ray powder diffraction pattern (XRD) of the crystal form E of the compound I were as shown in FIGS. 4-6. Specific peak values were as shown in Table 4.

TABLE 4

| Angle | d Value | Rel. |
|---|---|---|
| [°2Th.] | [Å] | Intensity [%] |
| 3.63 | 24.35 | 26.10 |
| 7.18 | 12.31 | 28.63 |
| 7.80 | 11.33 | 9.66 |
| 9.44 | 9.37 | 5.30 |
| 10.27 | 8.61 | 8.77 |
| 10.76 | 8.22 | 100.00 |
| 11.71 | 7.55 | 5.52 |
| 13.78 | 6.43 | 3.75 |
| 15.03 | 5.89 | 39.40 |
| 16.35 | 5.42 | 2.14 |
| 17.27 | 5.13 | 7.94 |
| 17.47 | 5.08 | 16.97 |
| 17.79 | 4.99 | 35.72 |
| 19.28 | 4.60 | 88.02 |
| 20.09 | 4.42 | 9.36 |
| 20.63 | 4.31 | 7.70 |
| 21.33 | 4.17 | 27.47 |
| 22.41 | 3.97 | 14.00 |
| 22.85 | 3.89 | 5.64 |
| 23.76 | 3.74 | 15.13 |
| 24.02 | 3.70 | 10.11 |
| 24.81 | 3.59 | 3.96 |
| 25.89 | 3.44 | 7.26 |
| 27.19 | 3.28 | 21.91 |
| 27.67 | 3.22 | 12.40 |
| 30.81 | 2.90 | 2.29 |

### Example 4: Preparation of Crystal Form F

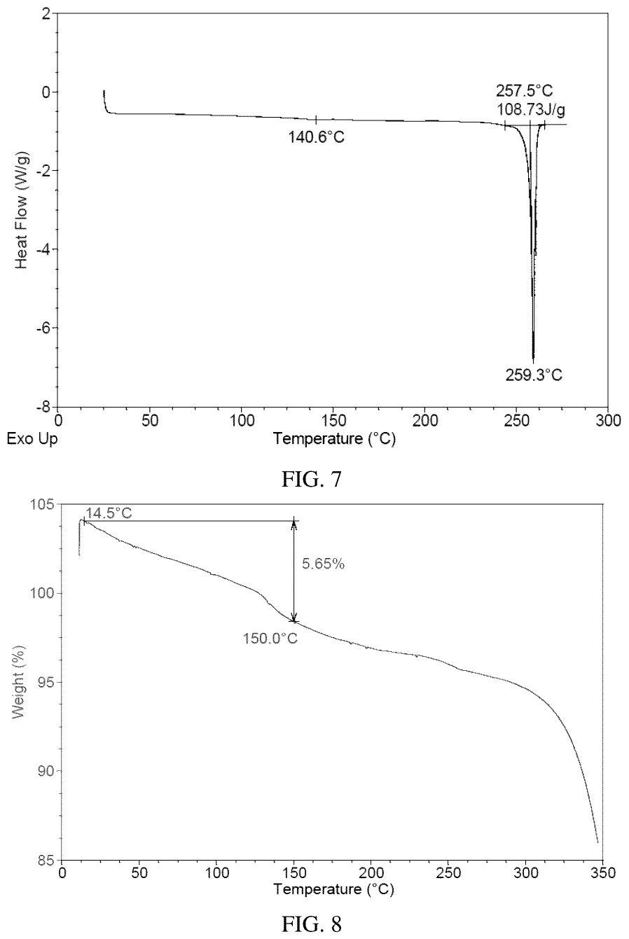

To the compound of formula I (50 mg), THF/H₂O (1 ml, 2:1, v/v) was added, and 1,4-dioxane (5 mL) was added dropwise. The mixture was exposed to an atmosphere at room temperature and slowly volatilized, to afford crystal form F. The differential scanning calorimetry curve pattern, thermogravimetric analysis pattern and X-ray powder diffraction pattern (XRD) of the crystal form F of the compound I were as shown in FIGS. 7-9. Specific peak values were as shown in Table 5.

TABLE 5

| Angle | d Value | Rel. |
|---|---|---|
| [°2Th.] | [Å] | Intensity [%] |
| 9.66 | 9.15 | 18.02 |
| 10.70 | 8.27 | 10.73 |
| 14.24 | 6.22 | 14.14 |
| 17.34 | 5.11 | 2.63 |
| 19.26 | 4.61 | 100.00 |
| 21.11 | 4.21 | 61.79 |
| 22.10 | 4.02 | 19.25 |
| 24.77 | 3.59 | 6.65 |

### Example 5: Preparation of Crystal Form G

To the compound of formula I (50 mg), MeOH (1 ml) was added, and the mixture was stirred at room temperature for 3 days, and then filtered and dried, to afford crystal form G. The differential scanning calorimetry curve pattern, thermogravimetric analysis pattern and X-ray powder diffraction pattern (XRD) of the crystal form G of the compound I were as shown in FIGS. 10-12. Specific peak values were as shown in Table 6. According to comprehensive analysis such as TGA, crystal form G is an anhydrous crystal form.

TABLE 6

| Angle | d Value | Rel. |
|---|---|---|
| [°2Th.] | [Å] | Intensity [%] |
| 7.81 | 11.32 | 100.00 |
| 9.47 | 9.34 | 33.47 |
| 10.30 | 8.59 | 62.46 |
| 11.70 | 7.56 | 50.85 |
| 12.37 | 7.16 | 24.63 |
| 17.27 | 5.13 | 5.87 |
| 19.44 | 4.57 | 57.95 |
| 19.75 | 4.50 | 14.59 |
| 20.03 | 4.43 | 16.68 |
| 20.41 | 4.35 | 25.87 |
| 20.66 | 4.30 | 66.06 |
| 21.18 | 4.19 | 5.64 |
| 21.60 | 4.11 | 6.25 |
| 22.49 | 3.95 | 24.01 |
| 26.38 | 3.38 | 6.75 |
| 26.77 | 3.33 | 11.00 |
| 27.50 | 3.24 | 6.90 |
| 28.96 | 3.08 | 7.99 |
| 33.69 | 2.66 | 1.85 |
| 37.22 | 2.42 | 1.14 |

### Example 6: Preparation of Crystal Form H

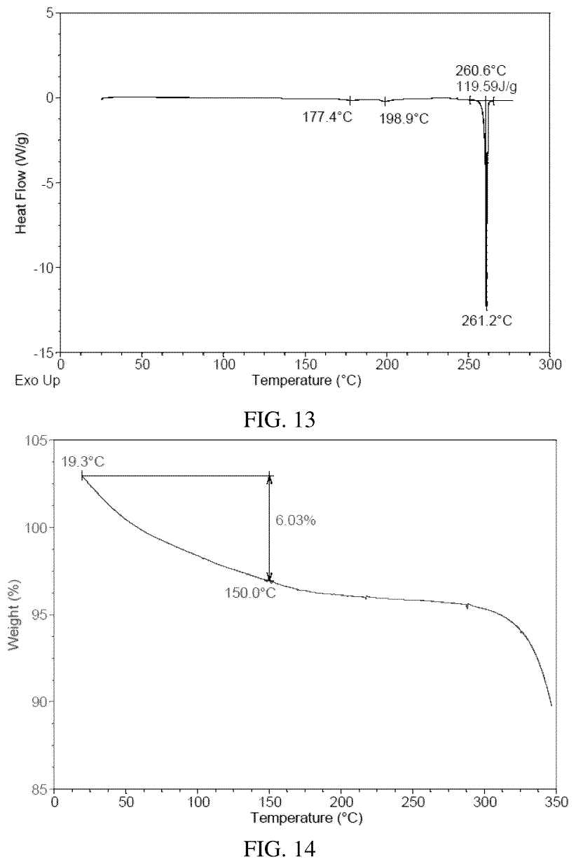

To the compound of formula I (50 mg), NMP was added until a clear solution was obtained, then MTBE was added dropwise until a solid precipitated. The mixture was continuously stirred for 1-2 h, filtered, dried at room temperature for 32 h, and dried under vacuum at 50° C. for 6 h, to afford crystal form H. The differential scanning calorimetry curve pattern, thermogravimetric analysis pattern and X-ray powder diffraction pattern (XRD) of the crystal form H of the compound I were as shown in FIGS. 13-15. Specific peak values were as shown in Table 7.

TABLE 7

| Angle | d Value | Rel. |
|---|---|---|
| [°2Th.] | [Å] | Intensity [%] |
| 5.11 | 17.29 | 29.52 |
| 8.73 | 10.12 | 15.75 |
| 10.39 | 8.52 | 5.16 |
| 15.92 | 5.57 | 13.72 |
| 16.99 | 5.22 | 12.40 |
| 17.34 | 5.11 | 21.94 |
| 18.07 | 4.91 | 10.75 |
| 21.09 | 4.21 | 100.00 |
| 23.46 | 3.79 | 4.58 |
| 24.79 | 3.59 | 10.75 |
| 25.49 | 3.49 | 7.01 |
| 26.33 | 3.39 | 6.93 |

### Biological Test Examples

### 1. PARP1 Enzyme Activity Test Experiment

PARP1 chemical fluorescence detection kit was purchased from BPS Bioscience. The histone solution in the kit was diluted 5X with 1×PBS, and 25 μL of the diluted histone solution was added to a microwell plate and incubated overnight at 4° C. After the incubation, the plate was washed three times with PBST (0.05% Tween-20). 100 μL of the blocking solution was added to the microwell plate and incubated at 25° C. for 90 minutes. After the incubation was completed, the plate was washed three times with PBST. 2.5 μL of compounds at various concentrations diluted in test buffer and 12.5 μL of substrate mixed solution (1.25 μL 10X PARP test buffer; 1.25 μL 10X PARP test mixed solution; 2.5 μL Activated DNA, 7.5 μL double-distilled water) were added to the microwell plate. The PARP1 enzyme was diluted to 2 ng/μL, 10 μL of the diluent was added to the microwell plate, and the reaction system was incubated at 25° C. for 60 minutes.

After the incubation was completed, the plate was washed three times with PBST. Streptavidin-HRP was diluted 50X with a blocking solution, and then 25 μL of the diluent was added to the microwell plate and incubated at 25° C. for 30 minutes. After the incubation, the plate was washed three times with PBST. ELISA ECL substrate A and substrate B were mixed at a ratio of 1:1 (v/v), 50 μL of the mixture was added to the microwell plate, and the chemiluminescence value was read.

The inhibition rate was calculated according to formula [(1−(RLUₛₐₘₚₗₑ−RLUₘᵢₙ)/(RLUₘₐₓ−RLUₘᵢₙ))×100%], where RLUsample was the readout of the compound well, RLUmax was the readout of the solvent control well, and RLUmin was the readout of the control well without the PARP1 enzyme. Curve fitting was performed by four parameters (log (inhibitor) vs. response—Variable slope) using GraphPad Prism software, and the IC₅₀ value was calculated.

TABLE 8

| Compound | IC₅₀ (nM) |
|---|---|
| I | 0.67 |

### 2. PARP2, PARP5A, PARP5B, PARP6, PARP7, PARP14 and PARP15 Enzyme Activity Test Experiments

PARP2, PARP5A, PARP5B, PARP6, PARP7, PARP14 and PARP15 chemical fluorescence detection kits were purchased from BPS Bioscience. The histone solution in the kit was diluted 5X with 1×PBS, and 25 μL of the diluted histone solution was added to a microwell plate and incubated overnight at 4° C. After the incubation, the plate was washed three times with PBST (0.05% Tween-20). 100 μL of the blocking solution was added to the microwell plate and incubated at 25° C. for 90 minutes. After the incubation was completed, the plate was washed three times with PBST. 2.5 μL of compound 1 diluted in test buffer and 5 μL of substrate mixed solution were added to the microwell plate. 5 μL of the diluted PARP enzyme was added to the microwell plate, and the reaction system was incubated at 25° C. for 60 minutes.

After the incubation was completed, the plate was washed three times with PBST. Streptavidin-HRP was diluted 50X with a blocking solution, and then 25 μL of the diluent was added to the microwell plate and incubated at 25° C. for 30 minutes. After the incubation, the plate was washed three times with PBST. ELISA ECL substrate A and substrate B were mixed at a ratio of 1:1 (v/v), 25 μL of the mixture was added to the microwell plate, and the chemiluminescence value was read.

The inhibition rate was calculated according to formula [(1-(RLUsample-RLUmin)/(RLUmax-RLUmin))×100%], where RLUsample was the readout of the compound well, RLUmax was the readout of the solvent control well, and RLUmin was the readout of the control well without the PARP1 enzyme. Curve fitting was performed by four parameters (log (inhibitor) vs. response--Variable slope) using GraphPad Prism software, and the IC₅₀ value was calculated.

Test results: The compound of the present invention had a weak inhibitory effect on PARP2 enzyme activity in vitro, and its corresponding IC₅₀ value was 27.47 nM; and the compound had a very weak inhibitory effect on PARP5A, PARP5B, PARP6, PARP7, PARP14 and PARP15 enzyme activities in vitro, and their corresponding IC₅₀ values were all greater than 500 nM. The specific test results were as shown in the following table.

TABLE 9

| Compound | PARP enzyme | IC₅₀ (nM) |
|---|---|---|
| I | PARP2 | 27.47 |
| I | PARP5A | 6076 |
| I | PARP5B | 576 |
| I | PARP6 | 6860 |
| I | PARP7 | 5356 |
| I | PARP14 | 7064 |
| I | PARP15 | 4304 |

The compound of the present invention has good PARP1 inhibitory selectivity.

### 3. MDA-MB-436 Cell Activity Test Experiment

Human breast cancer cells MDA-MB-436 were purchased from ATCC, placed in Leibovitz's L-15 medium (supplemented with 10 μg/mL insulin, 16 μg/mL glutathione, 10% fetal bovine serum and 1% penicillin-streptomycin solution) and cultured at 37° C. in a CO₂-free incubator. On day 1, the cells in the exponential growth phase were collected, and the cell suspension was adjusted to 4000 cells/135 μL with the culture medium. 135 μL of the cell suspension was added to each well of a 96-well cell culture plate and incubated overnight. On day 2, compounds at different concentrations were added, and the plate was placed in the incubator and incubated for 7 days. After the incubation was completed, according to operation instructions for a CellTiter-Glo kit (Promega, G7573), 75 μL of CTG solution, which had already been pre-melted and equilibrated to room temperature, was added to each well, and the mixture was uniformly mixed for 2 min using a microplate shaker. The plate was placed at room temperature for 10 min, and then fluorescence signal values were measured using an Envision2104 plate reader (PerkinElmer). The inhibition rate was calculated according to the formula [(1−(RLU_{compound}−RLU_{blank})/(RLU_{control}−RLU_{blank}))×100%], where RLU_{compound} was the readout of the drug treated group, RLU_{control} was the average value of the solvent control group, and RLU_{blank} was the average value of the cell-free well. The IC₅₀ value was calculated using GraphPad Prism software.

Test results: the compound of the present invention has a significant inhibitory effect on breast cancer cells MDA-MB-436.

TABLE 10

| Compound | IC₅₀ (nM) | Max inh. % 10 μM |
|---|---|---|
| I | 2 | 89.4 |

### 4. Mouse MDA-MB-436 Subcutaneous In Vivo Transplanted Tumor Model

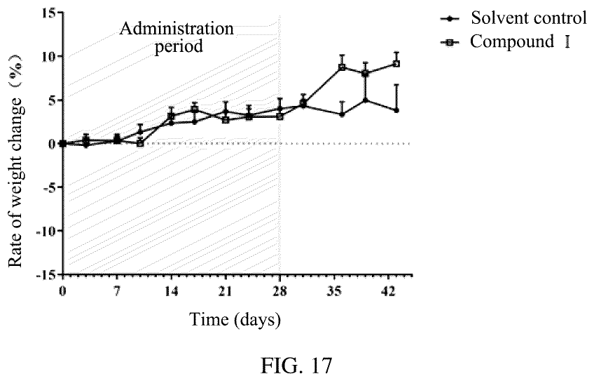

Human breast cancer cells MDA-MB-436 were placed in Leibovitz's L-15 medium (supplemented with 10 μg/mL insulin, 16 μg/mL glutathione, 10% fetal bovine serum and 1% penicillin-streptomycin solution) and cultured at 37° C. Conventional digestion treatment with trypsin was performed twice a week for passage. When the cell saturation was 80%-90%, and the number reached the requirement, the cells were harvested, counted, and inoculated. BALB/c nude mice (from Beijing Vital River Laboratory Animal Technology Co., Ltd.) were subcutaneously inoculated with 0.2 mL (10×10⁶) of MDA-MB-436 cells (plus matrigel, with the volume ratio of 1:1) on the right back. When the average tumor volume reached about 180 mm³, grouping and administration were performed (marked as Day 0). The solvent group was given 5% DMSO, 30% PEG400 and 65% of 20% sulfobutyl-β-cyclodextrin solution, and the administration group was given the compound (Day 0-Day 10:1 mg/kg; Day 11-Day 28:0.1 mg/kg). The administration frequency was once a day, the administration cycle was 29 days, and the drug withdrawal observation period was set to 14 days. After grouping, the tumor diameter was measured twice a week with a vernier caliper. The formula for calculating the tumor volume was: V=0.5×a×b², where a and b represented the long and short diameters of the tumor, respectively. The tumor inhibitory effect of the compound was evaluated by TGI (%)=[1−(average tumor volume at the end of administration in the treatment group-average tumor volume at the beginning of administration in the treatment group)/(average tumor volume at the end of treatment in the solvent control group-average tumor volume at the beginning of treatment in the solvent control group)]×100%. The tumor growth curve and the animal body weight change curve were as shown in FIG. 16 and FIG. 17, respectively.

Test results: after 28 days of administration, the TGI of the group given the compound was 119%; after the drug withdrawal, the tumor of the animals given the compound did not grow again; and there was no significant decrease in the body weight of the animals given the compound. This illustrates that the compound has good efficacy in inhibiting tumor growth and inducing tumor regression, and is well tolerated.

### 5. Pharmacokinetic Test in Rats

1.1 Experimental animals: male SD rats, about 220 g, 6-8 weeks old, 6 rats/compound. Purchased from Chengdu Ddossy Experimental Animals Co., Ltd.
1.2 Experimental design: on the day of the experiment, 6 SD rats were randomly grouped according to their body weights. The animals were fasted with water available for 12 to 14 h one day before the administration and were fed 4 h after the administration.

TABLE 11

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | samples | administration |
| G1 | 3 | Compound I | 2.5 | 0.5 | 5 | Plasma | Intravenous |
| | | | | | | | administration |
| G2 | 3 | | 10 | 1 | 10 | Plasma | Intragastric |
| | | | | | | | administration |

Notes:
Solvent for intravenous administration: 10% DMA + 10% Solutol + 80% Saline; solvent for intragastric administration: 5% DMSO + 30% PEG400 + 65% (20% SBE-CD) (DMA: dimethylacetamide; Solutol: polyethylene glycol-15-hydroxystearate; Saline: physiological saline; DMSO: dimethyl sulfoxide; SBE-CD: β cyclodextrin)

Before and after the administration, 0.15 mL of blood was taken from the orbit of the animals under isoflurane anesthesia and placed in an EDTAK2 centrifuge tube. The blood was centrifuged at 5000 rpm and 4° C. for 10 min to collect plasma. The blood collection time points for the intravenous administration group and intragastric administration group were: 0, 5 min, 15 min, 30 min, 1 h, 2 h, 4 h, 6 h, 8 h and 24 h. Before analysis and detection, all samples were stored at −80° C. The samples were analyzed quantitatively by LC-MS/MS. The test results of some examples were as shown below.

TABLE 12

| | Mode of | | | | |
|---|---|---|---|---|---|
| Test | admin- | CL | Vdss | AUC0-t | F |
| compound | istration | (mL/min/kg) | (L/kg) | (hr*ng/mL) | (%) |
| Com- | i.v. | 0.896 ± | 0.236 ± | 46601 ± | — |
| pound I | (2.5 mg/kg) | 0.097 | 0.022 | 4753 | |
| | i.g. | — | — | 184171 ± | 98.8 ± |
| | (10 mg/kg) | | | 72260 | 39 |

—: not applicable.

Conclusion: the compound has good pharmacokinetic characteristics in rats.

### Crystal Form Test Examples

### Stability Test

Samples were taken and tested at 92.5% RH, 40° C. and 60° C. respectively. The purity (represented by a percentage) was detected by HPLC. The experimental results were as shown in Table 15.

With regard to methods for preparing test solutions and conditions for detecting purity by HPLC, reference was made to Tables 13 and 14.

TABLE 13
Preparation method for test solution

| | |
|---|---|
| Diluent | Methanol |
| Blank | Methanol |
| solution | |
| Test | An appropriate amount of the samples was |
| solution | weighed precisely, and dissolved and diluted |
| | with methanol to prepare a solution containing |
| | about 0.5 mg of the sample per 1 ml. |

TABLE 14
Conditions for detecting purity by HPLC

| Instrument | Shimadzu Liquid Chromatograph LC-20AT | | |
|---|---|---|---|
| Chromatographic | Octadecyl silane bonded silica gel was used | | |
| column | as the filler (YMC Triart C₁₈ 4.6 × 250 mm, | | |
| | 5 μm, or an equivalent chromatographic column) | | |
| Mobile phase | Mobile phase A: disodium hydrogen phosphate | | |
| | buffer (0.02 mol/L); | | |
| | Mobile phase B: acetonitrile; | | |
| Detection | 220 nm | | |
| wavelength | | | |
| Flow rate | 1.0 ml/min | | |
| Column | 35° C. | | |
| temperature | | | |
| Injection volume | 10 μl | | |
| Acquisition time | 60 min | | |
| | Time | Mobile phase | Mobile phase |
| | (min) | A (%) | B (%) |
| Elution | 0 | 85 | 15 |
| procedure | 35 | 60 | 40 |
| | 50 | 40 | 60 |
| | 51 | 85 | 15 |
| | 60 | 85 | 15 |

Notes
The system is added to a trap cartridge.

TABLE 15
Chemical stability of crystal form G under different conditions (purity was determined by HPLC)

| | RRT | | | | | | | | |
|---|---|---|---|---|---|---|---|---|---|
| | | | | | | | | Maximum | |
| | | | | | | | | individual | Total |
| Condition | 0.31 | 0.36 | 0.49 | 0.72 | 1.14 | 1.33 | 1.00 | impurity | impurity |
| 0 d | 0.494% | 0.071% | 0.170% | 0.248% | 0.065% | 0.079% | 98.425% | 0.494% | 1.575% |
| 40° C.-6 d | 0.438% | 0.097% | 0.195% | 0.241% | 0.071% | 0.069% | 98.494% | 0.438% | 1.506% |
| 60° C.-6 d | 0.452% | 0.097% | 0.193% | 0.229% | 0.070% | 0.064% | 98.522% | 0.452% | 1.478% |
| 92.5% RH-6 d | 0.468% | 0.107% | 0.191% | 0.244% | 0.068% | 0.066% | 98.503% | 0.468% | 1.497% |
| 40° C.-10 d | 0.507% | 0.100% | 0.194% | 0.232% | 0.069% | 0.061% | 98.459% | 0.507% | 1.541% |
| 60° C.-10 d | 0.498% | 0.103% | 0.206% | 0.247% | 0.068% | 0.069% | 98.447% | 0.498% | 1.553% |
| 92.5% RH-10 d | 0.518% | 0.110% | 0.206% | 0.246% | 0.073% | 0.066% | 98.358% | 0.518% | 1.642% |
| 40° C.-30 d | 0.467% | 0.115% | 0.144% | 0.252% | 0.077% | 0.060% | 98.519% | 0.467% | 1.481% |
| 60° C.-30 d | 0.457% | 0.112% | 0.145% | 0.244% | 0.084% | 0.056% | 98.499% | 0.457% | 1.501% |
| 92.5% RH-30 d | 0.475% | 0.099% | 0.137% | 0.243% | 0.075% | 0.056% | 98.499% | 0.475% | 1.501% |

Notes:
RRT represents relative retention time.

Conclusion: crystal form G has good chemical stability.

1. A crystalline substance of a compound as shown in formula (I):

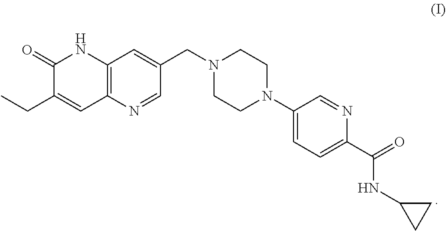 [SMILES: CCc1cc2ncc(CN3CCN(c4ccc(C(=O)NC5CC5)nc4)CC3)cc2[nH]c1=O]

2. The crystalline substance according to claim 1, wherein the crystalline substance is of crystal form B having an X-ray powder diffraction pattern with characteristic diffraction peaks at the following 2θ positions by using Cu-Kα radiation: 19.31°±0.2°, 20.37°±0.2°, 22.23°±0.2°, 22.90°±0.2°, 23.70°±0.2°, and 27.18°±0.2°.

3. The crystalline substance according to claim 2, wherein the crystalline substance has an X-ray powder diffraction pattern with characteristic diffraction peaks at the following 2θ positions: 11.12°±0.2°, 15.96°±0.2°, 16.93°±0.2°, 19.31°±0.2°, 20.37°±0.2°, 22.23°±0.2°, 22.90°±0.2°, 23.70°±0.2°, 25.45°±0.2°, 26.52°±0.2°, 27.18°±0.2°, 29.14°±0.2°, and 32.78°±0.2°.

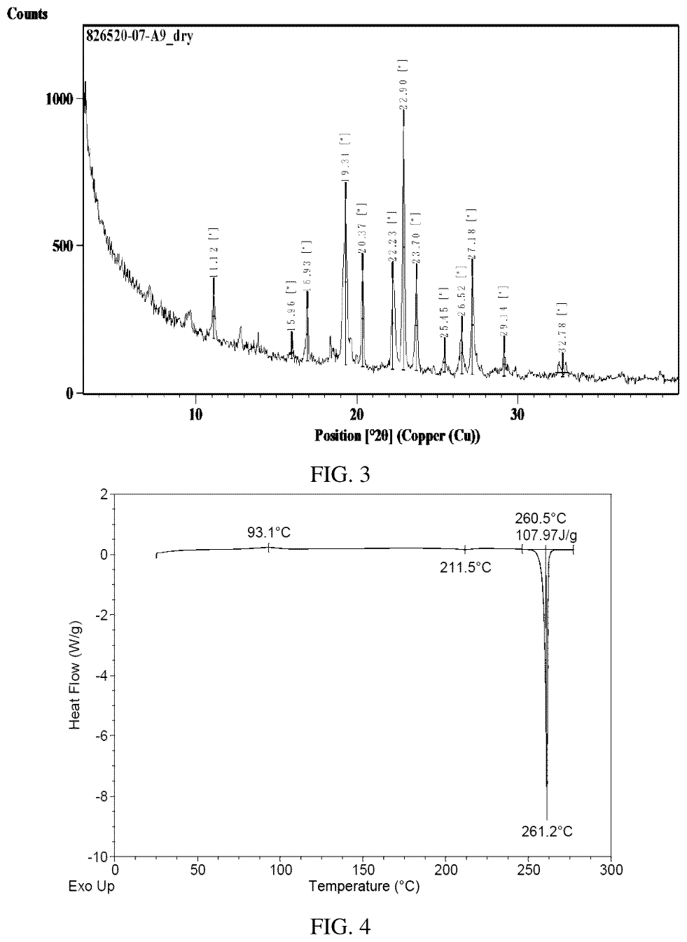

4. The crystalline substance according to claim 2, wherein the crystalline substance has an X-ray powder diffraction pattern substantially as shown in FIG. 3.

5. The crystalline substance according to claim 1, wherein the crystalline substance is of crystal form E having an X-ray powder diffraction pattern with characteristic diffraction peaks at the following 2θ positions by using Cu-Kα radiation: 3.63°±0.2°, 7.18°±0.2°, 10.76°±0.2°, 15.03°±0.2°, 17.47°±0.2°, 17.79°±0.2°, 19.28°±0.2°, 21.33°±0.2°, 23.76°±0.2°, and 27.19°±0.2°.

6. The crystalline substance according to claim 5, wherein the crystalline substance has an X-ray powder diffraction pattern with characteristic diffraction peaks at the following 2θ positions by using Cu-Kα radiation: 3.63°±0.2°, 7.18°±0.2°, 7.80°±0.2°, 10.27°±0.2°, 10.76°+0.2°, 15.03°±0.2°, 17.27°±0.2°, 17.47°±0.2°, 17.79°±0.2°, 19.28°±0.2°, 20.09°±0.2°, 20.63°+0.2°, 21.33°±0.2°, 22.41°±0.2°, 23.76°±0.2°, 24.02°±0.2°, 25.89°±0.2°, 27.19°±0.2°, and 27.67°±0.2°.

7. The crystalline substance according to claim 5- or 6, wherein the crystalline substance has an X-ray powder diffraction pattern substantially as shown in FIG. 6 by using Cu-Kα radiation.

8. The crystalline substance according to claim 1, wherein the crystalline substance is of crystal form F having an X-ray powder diffraction pattern with characteristic diffraction peaks at the following 2θ positions by using Cu-Kα radiation: 9.66°±0.2°, 10.70°±0.2°, 14.24°±0.2°, 17.34°±0.2°, 19.26°±0.2°, 21.11°±0.2°, 22.10°±0.2°, and 24.77±0.2°.

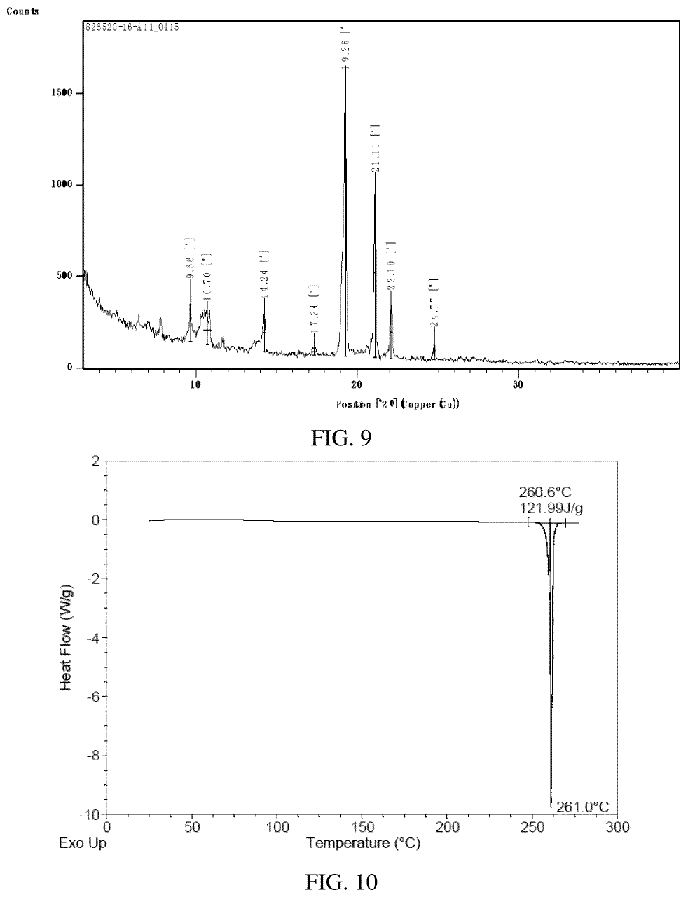

9. The crystalline substance according to claim 8, wherein the crystalline substance has an X-ray powder diffraction pattern substantially as shown in FIG. 9 by using Cu-Kα radiation.

10. The crystalline substance according to claim 1, wherein the crystalline substance is of crystal form G having an X-ray powder diffraction pattern with characteristic diffraction peaks at the following 2θ positions by using Cu-Kα radiation: 7.81°±0.2°, 9.47°±0.2°, 10.30°±0.2°, 11.70°±0.2°, 12.37°±0.2°, 19.44°±0.2°, 19.75°±0.2°, 20.03°±0.2°, 20.41°±0.2°, 20.66°±0.2°, 22.49°±0.2°, and 26.77°±0.2°.

11. The crystalline substance according to claim 10, wherein the crystalline substance has an X-ray powder diffraction pattern with characteristic diffraction peaks at the following 2θ positions by using Cu-Kα radiation: 7.81°±0.2°, 9.47°±0.2°, 10.30°±0.2°, 11.70°±0.2°, 12.37°±0.2°, 17.27°±0.2°, 19.44°±0.2°, 19.75°±0.2°, 20.03°±0.2°, 20.41°±0.2°, 20.66°±0.2°, 21.18°±0.2°, 21.60°±0.2°, 22.49°±0.2°, 26.38°±0.2°, 26.77°±0.2°, 27.50°±0.2°, 28.96°±0.2°, 33.69°±0.2°, and 37.22°±0.2°.

12. The crystalline substance according to claim 10 or 11, wherein the crystalline substance has an X-ray powder diffraction pattern substantially as shown in FIG. 12 by using Cu-Kα radiation.

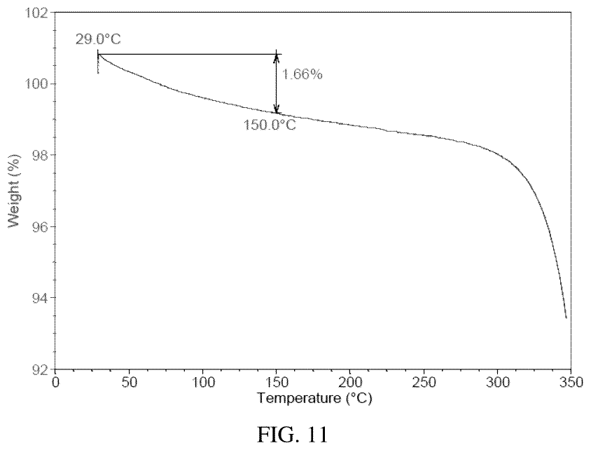

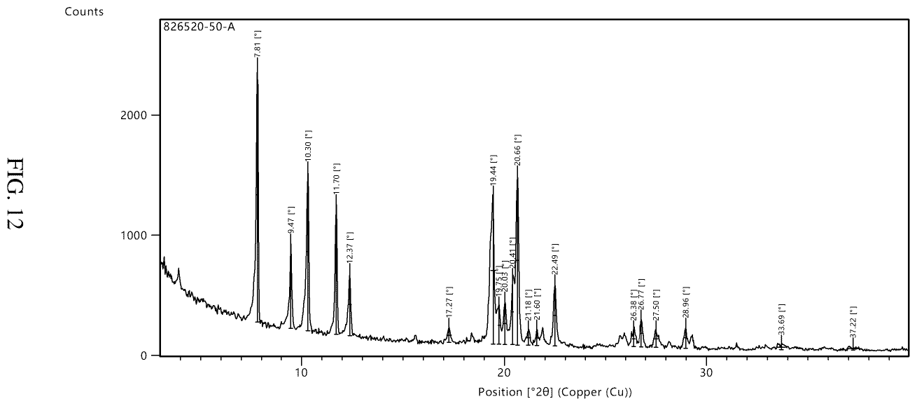

13. The crystalline substance according to claim 10, wherein the crystalline substance has a differential scanning calorimetry curve and a thermogravimetric analysis curve as shown in FIG. 10 and FIG. 11, respectively.

14. The crystalline substance according to claim 1, wherein the crystalline substance is of crystal form H having an X-ray powder diffraction pattern with characteristic diffraction peaks at the following 2θ positions by using Cu-Kα radiation: 5.11°±0.2°, 8.73°±0.2°, 10.39°±0.2°, 15.92°±0.2°, 16.99°±0.2°, 17.34°±0.2°, 18.07°±0.2°, 21.09°±0.2°, 23.46°±0.2°, 24.79°±0.2°, 25.49°±0.2°, and 26.33°±0.2°.

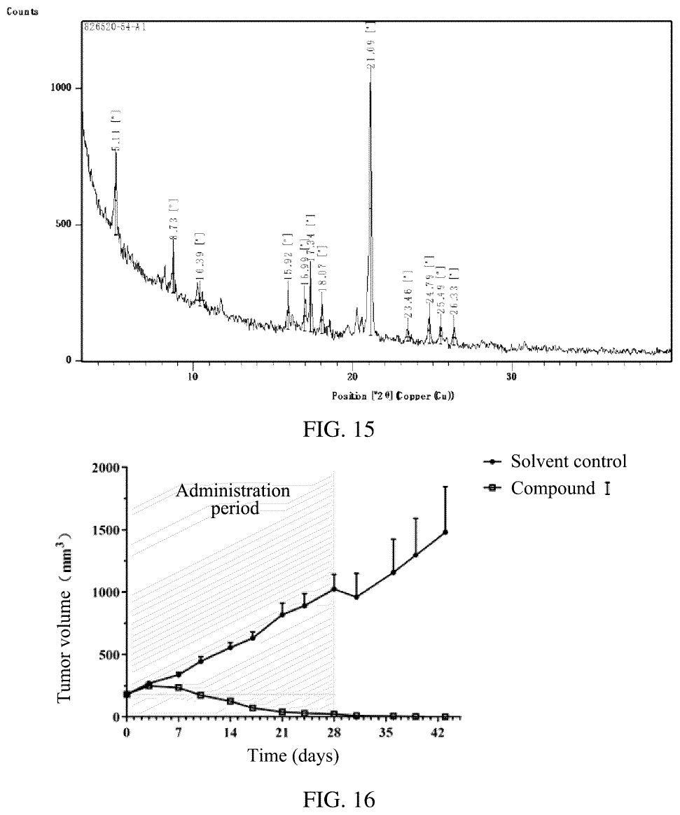

15. The crystalline substance according to claim 14, wherein the crystalline substance has an X-ray powder diffraction pattern substantially as shown in FIG. 15 by using Cu-Kα radiation.

16. A pharmaceutical composition, wherein the pharmaceutical composition comprises a therapeutically effective amount of the crystalline substance according to claim 1, and a pharmaceutically acceptable carrier and/or excipient.

17. (canceled)

18. A method for treating a disease in a mammal, comprising administering to a subject a therapeutically effective amount of the crystalline substance according to claim 1.

19. The pharmaceutical composition according to claim 16, wherein the therapeutically effective amount is 1-600 mg calculated based on a free base.

20. The method for treating a disease in a mammal according to claim 18, wherein the therapeutically effective amount is 1-600 mg calculated based on a free base.

21. The method for treating a disease in a mammal according to claim 18, wherein the disease is a tumor.
